SEQUENCE LISTING (3 sequences)

SEQ ID NO: 1 - DNA, 1419 bp, Homo sapiens
  “cDNA SELENBP1”
  cDNA SELENBP1
atggctacgaaatgtgggaattgtggacccggctactccacccctctggaggccatgaaaggacccagggaagagatcgtctacctgccctgcatttaccgaaacacaggcactgaggccccagattatctggccactgtggatgttgaccccaagtctccccagtattgccaggtcatccaccggctgcccatgcccaacctgaaggacgagctgcatcactcaggatggaacacctgcagcagctgcttcggtgatagcaccaagtcgcgcaccaagctggtgctgcccagtctcatctcctctcgcatctatgtggtggacgtgggctctgagccccgggccccaaagctgcacaaggtcattgagcccaaggacatccatgccaagtgcgaactggcctttctccacaccagccactgcctggccagcggggaagtgatgatcagctccctgggagacgtcaagggcaatggcaaagggggttttgtgctgctggatggggagacgttcgaggtgaaggggacatgggagagacctgggggtgctgcaccgttgggctatgacttctggtaccagcctcgacacaatgtcatgatcagcactgagtgggcagctcccaatgtcttacgagatggcttcaaccccgctgatgtggaggctggactgtacgggagccacttatatgtatgggactggcagcgccatgagattgtgcagaccctgtctctaaaagatgggcttattcccttggagatccgcttcctgcacaacccagacgctgcccaaggctttgtgggctgcgcactcagctccaccatccagcgcttctacaagaacgagggaggtacatggtcagtggagaaggtgatccaggtgccccccaagaaagtgaagggctggctgctgcccgaaatgccaggcctgatcaccgacatcctgctctccctggacgaccgcttcctctacttcagcaactggctgcatggggacctgaggcagtatgacatctctgacccacagagaccccgcctcacaggacagctcttcctcggaggcagcattgttaagggaggccctgtgcaagtgctggaggacgaggaactaaagtcccagccagagcccctagtggtcaagggaaaacgggtggctggaggccctcagatgatccagctcagcctggatgggaagcgcctctacatcaccacgtcgctgtacagtgcctgggacaagcagttttaccctgatctcatcagggaaggctctgtgatgctgcaggttgatgtagacacagtaaaaggagggctgaagttgaaccccaacttcctggtggacttcgggaaggagccccttggcccagcccttgcccatgagctccgctaccctgggggcgattgtagctctgacatctggatttga

SEQ ID NO: 2 - DNA, 37 bp, Artificial Sequence
  “Primer P1”
  Primer P1
atcggatccaccatggctacgaaatgtgggaattgtg

SEQ ID NO: 3 - DNA, 56 bp, Artificial Sequence
  “Primer P2”
  Primer P2
atcaagcttcagtgatggtgatggtgatgaatccagatgtcagagctacaatcgcc

(19) United States
(12) Patent Application Publication
(10) Pub. No.: US 2022/0082573 A1
(43) Pub. Date: Mar. 17, 2022

(54) SELENIUM BINDING PROTEIN 1 DETECTION FROM BODY FLUIDS FOR DIAGNOSIS OF PERACUTE TISSUE DAMAGE

(71) Applicant: berYsol GmbH, Berlin (DE)

(72) Inventors: Lutz Schomburg, Berlin (DE); Eike Christian Kuehn, Berlin (DE)

(73) Assignee: berYsol GmbH, Berlin (DE)

(21) Appl. No.: 17/418,189

(22) PCT Filed: Dec. 19, 2019
(86) PCT No.: PCT/EP2019/086474

§ 371 Date: Jun. 24, 2021

(30) Foreign Application Priority Data
Dec. 29, 2018 (DE) 10 2018 010 217.4

(51) Int. Cl. G01N 33/68

(52) U.S. Cl. CPC G01N 33/6893; G01N 2333/4703; G01N 2800/10

(57) ABSTRACT

The present invention refers to an immunoassay for determining the amount of selenium binding protein 1 (SELENBP1) in the body liquid of an individual suspected of suffering from peracute tissue damage and detecting pathologically elevated levels of SELENBP1, the diagnostic use of this immunoassay and a kit for the performance of a diagnostic assay comprising an antibody against SELENBP1.

The present invention refers to an immunoassay for determining the amount of selenium binding protein 1 (SELENBP1) in the body liquid of an individual suspected of suffering from peracute tissue damage and detecting pathologically elevated levels of SELENBP1, the diagnostic use of this immunoassay and a kit for the performance of a diagnostic assay comprising an antibody against SELENBP1.

Selenium (Se) is an essential trace element for mammals (Schwartz and Foltz (1958) J Biol Chem, 248-251). It is associated with the risk of developing type 2 diabetes mellitus (Rayman and Stranges (2013) Free Radic Biol Med, 65: 1557-1564), cancer (Clark, L. C. et al. (1996) JAMA 276, 1957-1963) and overall mortality (Rayman (2012) Lancet, 379: 1256-1268). There is a variety of biological forms in which selenium can be found in the human body. It is mostly incorporated in amino acids such as selenomethionine, selenocysteine and selenoneine, or in methylated amino acids such as methyl selenocysteine. It also occurs in inorganic compounds such as sodium selenite and selenate (Rayman (2012) Lancet, 379: 1256-1268). Bread, cereals and meat are the major dietary sources of selenium, but contents differ according to soil quality (Fairwearther-Tait et al. (2011) Antioxid Redox Signal, 14: 7).

There are two kinds of proteins associated with selenium in humans, selenoproteins and selenium binding proteins. Selenoproteins incorporate selenium in their primary structure in the form of selenocysteine. 25 human genes encoding for selenocysteine-containing proteins are know that give rise to ca. 100 gene products. This group of proteins comprises glutathione peroxidases (GPx), iodothyronine deiodinases and thioredoxin reductases which play a role in thyroid hormone metabolism, reactive oxygen species scavenging and redox state regulation in the cell (Schomburg (2012) Nat Rev Endocrinol 8: 160-171).

There are two selenium binding proteins, selenium binding protein 1 (SELENBP1; synonym: SP56) and selenium binding protein 2 (SELENBP2; synonym: acetaminophen-binding protein 56 (AP56). In humans only SELENBP1 has been identified so far.

SELENBP1 is a 56 kD monomeric protein. Its gene is located on chromosome 1q21-22 (Chang et al. (1997) J Cell Biochem 64: 217-224). Its amino acid sequence is known from UniProtKB database No. Q13228-1 and is encoded by SEQ:ID NO. 1 (cf., e.g., BC009084 (mRNA)). SELENBP1 can undergo cell type-specific tyrosine phosphorylation (cf. Torrealba et al. (2005) Am J Transplant 5: 58-67).

SELENBP1 expression was found to be down-regulated in inflamed tissue, e.g. in ulcerative colitis (Poulsen et al. (2012) BMC Gastroenterol 12: 76). SELENBP1 is the target of autoantibodies in Behcet's disease, a multitarget inflammatory disease (Okunuki et al. (2007) Exp Eye Res, 84: 823-831), and in women with premature ovarian failure, leading to infertility (Edassery et al. (2010) Fertil Steril 94: 2636-2641). SELENBP1 autoantibodies were also identified in ovarian disorders and ovarian cancer (Yi et al. (2017) Reproduction 153: 277-284). SELENBP1 was found to be decreased in twelve forms of cancer, e.g. in leiomyoma (Zhang et al. (2010) Diagn Pathol 5: 80). There are contradictory findings whether this reduction can be reversed by pharmaceutical antitumor treatment. It remains also illusive whether constantly increased or decreased SELENBP1 levels in tumor patients are a suitable predictor for a higher or lower survival rate.

US 2004/0170629 A1 discloses a method for monitoring smooth muscle abnormalities by assessing SELENBP1 levels in smooth muscle samples by means of mass spectroscopy and immunohistochemistry. It was found that in certain enduring and/or chronic inflammatory disease states such as acute or vascular chronic transplant rejection, arteriosclerosis, asthma, pregnancy complications associated with the uterus, and cancer SELENBP1 expression is absent or strongly down-regulated, compared to healthy persons. This down-regulation was taken as a diagnostic marker for smooth muscle cell abnormalities, particularly in transplant rejection.

In emergency medicine and intensive care units in hospitals it is essential to quickly assess the patient's current risk for a fatal or nearly fatal outcome as well as to establish a working diagnosis to prioritize diagnostic and therapeutic measures. In extreme cases every minute can count to save a patient's life. Standard diagnostic methods often take a significant amount of time to complete, results may be ambiguous and may require confirmation by means of further diagnostics. At times, the capacity of diagnostic devices, respectively the number of staff trained to handle them is limited. Furthermore, it is necessary to rule out false positive diagnoses in order to protect a patient from unnecessary diagnostic and therapeutic measures and emotional stress, as well as to keep healthcare costs in a reasonable range.

If a patient is suspected or diagnosed to suffer from a peracute, respectively fulminant pathological event the first medical priority is to systemically stabilize the patient's organism. The treatment of the specific pathology ensues afterwards.

A frequent peracute pathological event is myocardial infarction, histopathologically defined as the death of cardiomyocytes, i.e. heart muscle cells, due to prolonged ischemia, i.e. reduced blood flow. (Thygesen et al. (2018) Eur Heart J Vol. 39(42), pp. 3757-58; ehy462). The annual incidence is 1-5 per 1,000 inhabitants in industrialized countries. A myocardial infarction is often lethal if not attended quickly and treated adequately.

At present, Troponin T, Troponin I, and CK-MB (myocard-specific creatine kinase) serve as reliable serum markers indicative of myocardial injury. In particular, absolute levels or changes in levels of Troponin T and Troponin I as measured with a high sensitive assay have become a diagnostic gold standard in recent years. Serum levels of Troponin T and Troponin I increase within three to six hours after onset of symptoms. Hence a reliable diagnosis is not possible within this window and valuable time may pass before initiation of life-saving measures. Another limitation of the said tests according to the prior art is that Troponin T and/or Troponin I are released by skeletal and heart muscle cells and thus are at the same time insensitive in respect to other tissues and not completely specific in respect to cardiomyocytes. Therefore, peracute tissue damages that might have occurred in other organs or tissues cannot be detected with these tests.

Therefore, there is a medical need to overcome these problems. Thus, the task of the present invention is to provide a quick and easy-to-handle biochemical test able to indicate from an easy-to-get test sample such as blood, plasma, serum or urine, whether an admitted patient is suffering from such a peracute pathological damage, especially from an ACS or myocardial infarction. Desirably, such a test allows also for a quantitative estimation of the degree of the damage caused by a peracute pathological event. Furthermore, it is an alternative task of instant invention to provide a biochemical test for the presence of Alzheimer's disease. Such a biochemical test should enable a physician to make a well-founded decision whether it is necessary to stabilize the patient systemically and, if possible, to initiate a disease-specific treatment.

Surprisingly, it was found that the above-mentioned problems can be solved by an immunoassay against SELENBP1 in a test sample from body fluid of an individual.

It was found that upon a peracute tissue damage SELENBP1 is released from damaged cells into the circulation. Therefore, the detected amount of this protein can serve as a reliable biomarker indicative of an ongoing or very recent peracute tissue damage. The amount of SELENBP1 in such a test sample can be measured best by means of an immunochemical method. An antibody specifically binding to SELENBP1 is able to fulfill this task. This happens from a broad variety of tissues or organs in the organism of a mammal, though not in all tissues. The amount of liberated SELENBP1 is so high that the levels detected in test samples from body fluids such as blood, urine or liquor are unambiguously increased and thus distinguishable, in comparison to persons without such a peracute tissue damage (“healthy controls”).

As shown in the example section, SELENBP1 concentrations from patients diagnosed with Acute Coronary Syndrome (ACS) are drastically increased in comparison with a control group of healthy persons. ACS is a syndrome that becomes manifest with decreased blood flow in the coronary arteries. This way the heart muscle becomes unable to function properly and the heart muscle tissue is going to die off. ACS encompasses three major cardiac events: a) ST elevation myocardial infarction (STEMI), b) non-ST elevation myocardial infarction (NSTEMI), and c) unstable angina.

As SELENBP1 increased rapidly after the onset of ACS in patients with myocardial ischemia, SELENBP1 constitutes a novel and diagnostically helpful biomarker for patients having suffered from peracute tissue damage such as an ACS event.

It was found that the concentration of circulating SelenBP1 can be assessed best by means of a specific immunoassay. As described in Examples 4-5, monoclonal antibodies against SELENBP1 were produced and an immunoassay established.

Surprisingly, the task could be solved with the following immunochemical method:

An immunoassay for determining peracute tissue damage in a test sample from a subject suspected thereof, the immunoassay comprising the steps of:
a) Contacting a test sample taken from a subject suspected to suffering from peracute tissue damage with an antibody that binds specifically to at least one epitope of SELENBP1 to form an antibody:SELENBP1 complex;
b) Detecting the amount of antibody:SELENBP1 complex in said test sample; and
c) Assessing the degree of peracute tissue damage based on the detected amount of antibody:SELENBP1 complex.

Preferably, the immunoassays according to instant invention are for diagnostic purposes, especially for in-vitro diagnostic purposes.

In a preferred embodiment, the present invention refers to an immunoassay for determining peracute tissue damage in a test sample from a subject suspected of suffering from acute coronary syndrome, the immunoassay comprising the steps of:
a) Contacting a test sample taken from a subject suspected to suffering from peracute tissue damage due to ACS with an antibody that binds specifically to at least one epitope of SELENBP1 to form an antibody:SELENBP1 complex;
b) Detecting the amount of antibody:SELENBP1 complex in said test sample; and
c) Assessing the degree of peracute tissue damage based on the detected amount of antibody:SELENBP1 complex.

In a more preferred embodiment, the present invention refers to an immunoassay for determining peracute tissue damage in a test sample from a subject suspected of suffering from myocardial infarction, the immunoassay comprising the steps of:
a) Contacting a test sample taken from a subject suspected to suffering from peracute tissue damage due to myocardial infarction with an antibody that binds specifically to at least one epitope of SELENBP1 to form an antibody:SELENBP1 complex;
b) Detecting the amount of antibody:SELENBP1 complex in said test sample; and
c) Assessing the degree of peracute tissue damage based on the detected amount of antibody:SELENBP1 complex.

In other embodiments, the immunoassays according to instant invention comprise the step of calculating the amount of SELENBP1 based on the detected amount of antibody:SELENBP1 complex.

Alternatively, the method according to the invention can be described as follows:

A method of identifying a subject in need of medical intervention exhibiting peracute tissue damage, comprising the following steps:
a) Contacting a test sample taken from a subject suspected to suffering from peracute tissue damage with an antibody that binds specifically to at least one epitope of SELENBP1 to form an antibody:SELENBP1 complex in said test sample;
b) Detecting the amount of antibody:SELENBP1 complex;
c) Calculating the amount of SELENBP1 from step b);

wherein an amount of SELENBP1 equal or greater than 0.32 nmol/l, preferably equal or greater than 0.39 nmol/l in said undiluted test sample from said subject indicates that said subject is in need of a medical intervention.

In a preferred embodiment, the present invention refers to a method of identifying a subject in need of medical intervention exhibiting peracute tissue damage due to acute coronary syndrome, comprising the following steps:
a) Contacting a test sample taken from a subject suspected to suffering from peracute tissue damage due to acute coronary syndrome with an antibody that binds specifically to at least one epitope of SELENBP1 to form an antibody:SELENBP1 complex; in said test sample;
b) Detecting the amount of antibody:SELENBP1 complex;
c) Calculating the amount of SELENBP1 from step b);

wherein an amount of SELENBP1 equal or greater than 0.32 nmol/l, preferably equal or greater than 0.39 nmol/l in said undiluted test sample from said subject indicates that said subject is in need of a medical intervention.

In a more preferred embodiment, the present invention refers to a method of identifying a subject in need of medical intervention exhibiting peracute tissue damage due to myocardial infarction, comprising the following steps:
a) Contacting a test sample taken from a subject suspected to suffering from peracute tissue damage due to myocardial infarction with an antibody that binds specifically to at least one epitope of SelenBP1 to form an antibody:SELENBP1 complex; in said test sample;
b) Detecting the amount of antibody:SELENBP1 complex;
c) Calculating the amount of SELENBP1 from step b);

wherein an amount of SELENBP1 equal or greater than 0.32 nmol/l, preferably equal or greater than 0.39 nmol/l in said undiluted test sample from said subject indicates that said subject is in need of a medical intervention.

An alternative embodiment of the invention is the method of identifying a subject in need of medical intervention exhibiting peracute tissue damage, comprising the following steps:
a) Contacting a test sample taken from a subject suspected to suffering from peracute tissue damage with an antibody that binds specifically to at least one epitope of SELENBP1 to form an antibody:SELENBP1 complex in said test sample;
b) Detecting the amount of antibody:SELENBP1 complex;
c) Calculating the amount of SELENBP1 from step b);

wherein said test sample taken from a subject is urine or liquor; and wherein an amount of SELENBP1 equal or greater than 0.1 nmol/l, preferably equal or greater than 0.15 nmol/l, and most preferred equal or greater than 0.2 nmol/l in said undiluted test sample from said subject indicates that said subject is in need of a medical intervention.

Optionally, each of the four aforementioned methods may comprise after step c) the additional step: C′) Quantifying based on the detected amount of said SELENBP1 in said undiluted sample of body liquid of said subject the degree of peracute tissue damage in said subject.

The term “immunoassay” according to the present invention preferably means any immunochemical detection method in a test sample. In particular, the said term refers to a detection method of a protein or peptide by means of at least one suitable antibody against at least one epitope of said protein or peptide.

The term “subject” according to the present invention preferably means an individual, which is any mammal, preferably a human being, irrespectively of its health status. The terms “medicine”, “medication” or “medical” according to the present invention preferably encompass human medicine as well as veterinary medicine, especially human medicine.

There is an established classification of different time courses of disease states in medicine. There are peracute diseases, acute diseases, subacute diseases and chronic diseases. However, due to the heterogeneity of disease courses there are no generally accepted time intervals for all diseases. The most precise parameter is the onset of the disease to which will be referred to in the scope of the present application: Peracute diseases display often a very acute and violent disease course. Symptoms develop usually during a few hours. They are often, but not exclusively, due to an unusual traumatic event that causes drastic changes in the organism. In severe cases symptoms may be life-threatening. Acute diseases use to develop quickly and have often a short duration. In general, the term “acute” according to the present invention preferably means disease courses with a time frame of 1 to a few days. Subacute diseases refer to disease courses located between acute and chronic diseases. They become manifest after a few days and can take up to one week. Chronic diseases develop relatively slowly. They take minimum one week and can endure up to several months or years.

The term “tissue damage” according to the present invention preferably means any pathophysiological damage of cells or cell groups in a tissue that eventually leads to a loss of function of these cells or to a loss of these cells. This loss can be traumatically induced, i.e. a physical, chemical, electrical damage of the integrity of the cell, by over-excitation, inflammation, electroporation or irradiation, it can be necrotic or intrinsically or extrinsically apoptotic. The common denominator is that a tissue damage leads to a disability in functioning of the affected cells and a drastically altered metabolism and redox state. Optionally, these cells may finally enter a death-related pathway (apoptosis, necrosis, ferroptosis or similar) and die, the barrier between the cytosol and the extracellular fluid breaks down, respectively the cell membrane becomes significantly leaky, and cell debris and cytosolic components are released into the extracellular fluid and eventually to the circulation.

The term “peracute tissue damage” according to the present invention preferably means a pathophysiological event entailing tissue damage that occurs in the typical time frame of a peracute disease course as defined above. As a correlate of a peracute tissue damage the related cell signals, debris and/or the cytosolic components released into the circulation become detectable shortly after the underlying pathophysiological event, i.e. after a few hours.

The term “test sample” according to the present invention preferably means a sample of a body fluid from a mammal that is suspected to suffering from peracute tissue damage. In order to detect such peracute tissue damage with the immunoassay according to the invention a test sample such as blood, plasma, serum, urine or liquor has to be taken from said subject. According to the presently available methods, the volume of such a test sample is preferably about 1000 to 1 μl, preferably about 100 to 10 μl.

The term “pathologically elevated levels of SELENBP1” according to the present invention preferably means a concentration of SELENBP1 in said test sample that is unambiguously higher than in a healthy person or unambiguously elevated in respect to individual baseline values in a person with a chronic inflammatory disease due to suffered peracute tissue damage that has given rise to a pathologically elevated amount of SELENBP1 in the circulation.

The term “antibody (Ab)” according to the present invention preferably means a protein of the immunoglobulin (Ig) family. There are the subfamilies of IgA, IgD, IgE, IgG and IgM in mammals. They consist of two heavy and two light chains arranged in a Y-shaped manner. On the tips of the “Y” the Ab has a highly variable and antigen-specific region (Fab's variable region) which allows recognizing a specific epitope of the antigen. Via its paratope region which is specific for the antigen's epitope an antibody is able to bind these two regions together with precision. In an immunoassay the protein or peptide analyte is the antigen.

The term “antibody” according to the present invention preferably means a monoclonal antibody, a polyclonal antibody, a single chain antibody, a bispecific antibody or diabody, a bivalent antibody, a multispecific antibody, a synthetic antibody, an aptamer, a spiegelmer, a human or humanized antibody, and a fragment or variant thereof such as, e.g., Fab, Fv or scFv fragments, or a chemically modified derivative of any of these, e.g. antibody-drug conjugates, domain antibodies, nanobodies or antibody mimetica (DARPins, designed ankyrin repeat proteins). Most preferred are monoclonal antibodies due to their high specific and selective binding properties to SelenBP1 or equivalents with respect to their binding properties thereof. In another embodiment of the invention the antibody is directed to a SELENBP1 antigen, which is a physiological or pathophysiological derivative of natural occurring SELENBP1.

The term “analyte” according to the present invention preferably means any molecule with which an analyte antibody can interact and which is capable of binding an (one or more) analyte antibody to form specific complexes comprising [analyte antibody-antigenic molecule]. According to the present invention, the antigenic molecule, respectively analyte, is SELENBP1.

The term “specifically binding” according to the present invention preferably means that the antibody according to the invention does not bind substantially to (“cross-react” with) another protein, peptide or substance present in said test sample to be analyzed, which does not belong to SELENBP1. Preferably, the specifically bound protein should be bound with at least 3 times higher, more preferably 10 times higher and even more preferably 50 times higher affinity than any other relevant protein, peptide or substance. Nonspecific binding may be tolerable. It can still be distinguished and measured unequivocally, e.g. according to its size on a Western Blot, or by its relatively higher abundance in the test sample.

The term “amount” according to the present invention preferably means the absolute amount of the protein, the relative amount or concentration of said protein as well as any value or parameter which correlates thereto or can be derived therefrom. Such values or parameters comprise intensity signal values from all specific physical or chemical properties obtained from said protein by direct or indirect measurement. It is to be understood that values correlating to the aforementioned amounts or parameters can also be obtained by all standard mathematical operations.

The term “epitope”, respectively “epitope of SELENBP1” according to the present invention preferably means the specific part of the antigen, in the present case of the analyte SELENBP1, to which an antibody according to the invention binds. This epitope is a specific amino acid sequence of SELENBP1 that can be bound by said Ab. Depending on the specific antibody this epitope may vary. It is preferred that such an epitope allows for a specific binding of an antibody so that cross-reactivity with epitopes from other proteins can be avoided.

The term “antibody: SELENBP1 complex” according to the present invention preferably means the complex of SELENBP1 and an antibody of the invention bound to an epitope of SELENBP1. This term refers equally to the state of SELENBP1 being bound by an antibody according to instant invention.

The terms “identifying” and “identifying a subject in need of medical intervention” according to the present invention preferably means assessing whether a subject will be susceptible for a medical intervention or not. As it is understood by those skilled in the art such an assessment is usually not intended to be correct for a 100% of the subjects to be identified. It is, however, required that a statistically significant portion of subjects can be identified correctly. Whether a portion is statistically significant can be determined by a person skilled in the art using statistical methods well known in the art.

The term “detecting the amount of antibody:SELENBP1 complex” according to the present invention preferably means determining the amount or concentration, preferably quantitatively or relatively. This is to provide a direct or indirect means of the absolute or relative amount of SELENBP1 in the test sample, preferably together with calibration or standardization samples comprising a known amount of SELENBP1. The measuring can be done directly or indirectly. Direct measuring relates to measuring the amount or concentration of one or more of the reaction educts and/or the reaction products based on a signal which is obtained from the one or more reaction educts and/or the reaction products itself/themselves and the intensity of which directly correlates with the number of molecules of the one or more reaction educts and/or the reaction products in the reaction volume. Such a signal may be obtained, e.g., by measuring the intensity or value of a specific physical or chemical property of the one or more reaction educts and/or reaction products. Indirect measuring includes measuring of a signal obtained from a secondary component (i.e. a component not being the reaction educt or reaction product itself) or a biological read out system, referred to as “label means” in this specification, e.g., of measurable cellular or transmembrane responses, ligands, or enzymatic reaction products, e.g. by means of fluorophores, chromophores, ion concentrations, which suitably is performed by means of optical, electrical and/or electronical equipment. For measurement of enzymatic reaction products, preferably the amount of substrate is saturating. Optionally, the substrate may also be labeled with a detectable label prior to the reaction. Preferably, the reaction partners are contacted with the substrate for an adequate period of time, which corresponds to the time necessary for a detectable amount of the one or more reaction products to be produced such as a measurable signal. Instead of measuring the amount or concentration of the one or more reaction products, the time necessary for appearance of a given (e.g. detectable) amount or concentration of the one or more reaction products can be measured.

According to the present invention, the term “detecting the amount of antibody:SELENBP1 complex” can be preferably achieved by all means for determining the amount of a reaction educt and/or reaction product known to the skilled person. Said means comprise immunoassay devices and methods which may utilize labeled molecules in various sandwich, competition, or other assay formats. Said assays will develop a signal which is indicative for the presence or absence of the reaction educts and/or the reaction products. Moreover, the signal strength can, preferably, be correlated directly or indirectly (e.g. reverse-proportional) to the amount of reaction educts and/or the reaction products in the reaction volume. Said methods comprise, preferably, biosensors, optical devices coupled to immunoassays, biochips, analytical devices such as spectrometers or chromatography devices. Further, methods include ELISA-based methods using, optionally pre-treated or pre-coated, micro-plates, micro-arrays, or tube-arrays, fully-automated or robotic immunoassays (available, e.g., on Roche-Elecsys™, Abbott-AxSYM™ or Brahms Kryptor™ analyzer systems). Preferably, the term “detecting the presence and/or the binding properties” according to instant invention comprises the steps which will allow bringing the reaction partners together for an adequate period of time.

The term “in need of a medical intervention” according to the present invention preferably means that said subject exhibits symptoms and/or physical signs known to be associated with a severe peracute pathological event.

The term “medical intervention” according to the present invention preferably encompasses any prophylactic and/or therapeutic treatment regime that is found apt in the art to prevent or treat all peracute, acute and/or chronic damage in the subject due of a specific pathologic event as well as to prevent or treat all individual suffering of said subject due of this specific pathologic event. It can encompass any pharmaceutical, surgical, physiotherapeutic, dietary or psychological method known in the art to be beneficial for such a patient in need of a medical intervention.

The term “assessing the degree of peracute tissue damage” according to the present invention preferably means a medical evaluation whether the amount of antibody:SELENBP1 complex is indicative that a medical treatment should be started. Said amount may also be indicative which scope of treatment is medically needed and in which time frame it has to be started. A threshold for this amount may vary according to the suspected peracute tissue damage-causing pathological event and to the general constitution of said subject.

The term “diagnosing based on the detected amount of antibody:SelenBP1 complex” according to the present invention preferably means that a medically qualified person, preferably a physician, but also a nurse or a paramedic, can take a reason-based decision whether the patient from which the sample has been analyzed is in need of an acute medical intervention. Preferably, also the severity degree can be estimated, respectively diagnosed.

However, it is also known that during chronic inflammatory diseases there is also a continuous tissue damage which in most cases occurs in a more or less controlled manner.

This tissue damage may be overcome or counterbalanced by tissue regeneration or healing processes. Hence there is also a continuous cell loss in chronic inflammatory or ischemic diseases. Thus, there is a steady amount of SELENBP1 released into the circulation which can also be detected by the immunoassay of the present invention. However, the amounts detected in the respective test sample are by far below the values of the amount of SelenBP1 found in patients who have suffered peracute tissue damage. Therefore, the immunoassay according to the invention does not bear the risk of wrongly diagnosing a disease entailing peracute tissue damage instead of a chronic inflammatory disease or a minor tissue damage.

It has been observed that in some rare pathological cases autoantibodies against SELENBP1 can be found, e.g. in subgroups of ovarian disorders such as premature ovarian failure (POF) or irregular ovulation and subgroups of ovarian cancer (Yi et al. (2017) Reproduction 153:277-284). These autoantibodies against SELENBP1 might theoretically bias the quantitative detection of the immunoassay of the invention by binding circulating SELENBP1 and thus detract them from detection by the diagnostic SELENBP1 antibody of the invention, thereby wrongly diminishing the detected SELENBP1 concentration in the test sample. However, serum levels of autoantibodies are in general rather low so that they often escape detection. This is also the case in these reported SELENBP1 autoantibodies. The indicated OD (optical density) levels corresponding to the SELENBP1 concentrations are hardly elevated in comparison with healthy persons or with women with ovarian diseases in which no autoantibodies could be observed. Thus, in the very rare cases of such an autoantibody disease and a coincidental pathological event entailing a peracute tissue damage the bias would be minimal, if at all.

Thus, in a further embodiment of the method of the present invention it is additionally required that peracute tissue damage is diagnosed by the immunoassay of the invention if the amount of SELENBP1 in said test sample is higher than 0.32 nmol/l, preferably higher than 0.35 nmol/l.

In particular, the immunoassay according to the present invention preferably refers to an immunoassay, wherein the suspected peracute tissue damage is due to a peracute hypoxic event, a soft tissue trauma, acute liver failure, a fulminant soft tissue disease, a fulminant intoxication, a fulminant infection and/or Alzheimer's disease.

Hypoxemia must be differentiated from hypoxia. While hypoxia describes a state in which the organism, an organ or a tissue are continuously exposed to a reduced oxygen supply hypoxemia describes an abnormally low oxygen partial pressure in the blood, in particular in the arterial blood.

The term “peracute hypoxic event” according to the present invention preferably means any pathologic event in the organism that is causal, coincidental or the consequence of a hypoxia. This event may have become manifest systemically or locally. Such diseases or disease states comprise, without being limiting, coronary artery disease, ST elevation myocardial infarction (STEMI), non ST elevation myocardial infarction (NSTEMI), unstable angina, acute myocardial infarction, acute transmural myocardial infarction of anterior wall, acute transmural myocardial infarction of inferior wall, acute transmural myocardial infarction of other sites, acute transmural myocardial infarction of unspecified site, acute subendocardial myocardial infarction, acute myocardial infarction (unspecified), subsequent myocardial infarction, subsequent myocardial infarction of anterior wall, subsequent myocardial infarction of inferior wall, subsequent myocardial infarction of other sites, subsequent myocardial infarction of unspecified site, haemopericardium following acute myocardial infarction, atrial septal defect following acute myocardial infarction, ventricular septal defect following acute myocardial infarction, rupture of cardiac wall without haemopericardium following acute myocardial infarction, rupture of chordae tendineae following acute myocardial infarction, rupture of papillary muscle following acute myocardial infarction, thrombosis of atrium, auricular appendage and ventricle following acute myocardial infarction, coronary thrombosis not resulting in myocardial infarction, Dressler syndrome, acute ischemic heart failure (unspecified), pulmonary embolism, pulmonary embolism with mention of acute cor pulmonale, pulmonary embolism without mention of acute cor pulmonale, rupture of pulmonary vessel, stenosis of pulmonary vessel, stricture of pulmonary vessel, atrioventricular block, left anterior fascicular block, left posterior fascicular block, left bundle-branch block, right fascicular block, bifascicular block, trifascicular block, nonspecific intraventricular block, sinoatrial block, sinoauricular block, heart block not otherwise specified (NOS), Stokes-Adams syndrome, cardiac arrest with successful resuscitation, ventricular fibrillation and flutter, congestive heart failure, right ventricular failure, left ventricular failure, pulmonary edema with mention of NOS heart failure, intracardiac thrombosis (apical, atrial, auricular, ventricular), arterial embolism, arterial thrombosis, embolism and thrombosis of abdominal aorta, embolism and thrombosis of arteries of upper extremities, embolism and thrombosis of arteries of lower extremities, embolism and thrombosis of iliac artery, stricture of artery, rupture of artery, venous embolism, venous thrombosis, portal vein thrombosis, Budd-Chiari syndrome (fulminant hepatic venous thrombosis), embolism and thrombosis of vena cava, embolism and thrombosis of renal vein, thrombophlebitis migrans, thrombophlebitis, thrombophlebitis of superficial vessels of lower extremities, thrombophlebitis of deep vessels of lower extremities, thrombophlebitis of femoral vein, acute pulmonary insufficiency following thoracic surgery, acute pulmonary insufficiency following non-thoracic surgery, acute hypoxemic respiratory failure (AHRF), cardiogenic pulmonary edema (CPE), acute respiratory distress syndrome (ARDS), intraoperative hypoxemia, insufficient alveolar ventilation, epileptic seizures affecting the respiratory control, cervical neck fracture, carbon monoxide intoxication, suffocation, altitude sickness, freediving blackout, heart ischemia, atheromatous stenosis, disseminated intravascular coagulation.

A trauma (injury) is a damage caused to the body by external force. It may be caused by accidents, surgery, weapons, falls, hits, asphyxia, bites, stings or other incidents with animals or other causes. It may entail external and/or internal injuries. They can be inflicted by another person, animal or self-inflicted. In the scope of the present application the term trauma shall also encompass a burn, a scald, tissue damage by chemical substances including gases or a radiation injury. It is understood that a trauma leading to elevated SELENBP1 levels in a test sample must be serious enough to demand immediate medical attention. Minor traumas are not addressed by the immunoassay according to the invention.

Acute liver failure (fulminant liver failure) is characterized as a severe complication with a rapid onset after liver disease. It is associated with a loss of function of about 80-90% of the liver cells. In a majority of cases acute liver failure is due to systemic inflammatory syndrome (SIRS) that can eventually lead to multi-organ failure. A major cause therefore is bacterial and/or fungal sepsis. Common causes include medication overdose, idiosyncratic reaction to medication, excessive alcohol and/or drug consumption, fulminant viral hepatitis A and B, acute fatty liver of pregnancy, Reye syndrome, Wilson's disease, mushroom intoxication such as death cap, or it may be idiopathic.

Acute renal failure is often a consequence of exsiccosis, medication, arterial hypotension either traumatic, iatrogenic or due to SIRS with or without sepsis or idiopathic, fulminant liver failure or of fulminant diseases of the biliary tract, or of rhabdomyolysis. It becomes manifest within a short delay of time. There are also cases of fulminant postpartum renal failure or due to severe inner hemorrhages, rapidly progressing glomerulonephritis (RPGN), renal papillary necrosis, emphysematous pyelonephritis, and others.

Soft tissue includes the tissues that connect, support, or surround other structures and organs of the body, not being hard tissue such as bone. Soft tissue includes tendons, ligaments, fascia, skin, fibrous tissues, fat, synovial membranes, muscles, nerves and blood vessels. There is a variety of fulminant soft tissue diseases comprising without being limiting, compartment syndrome, acute fulminant necrotizing lymphocytic myocarditis, severe necrotizing soft tissue disease, clostridial myonecrosis, acute lupus pneumonitis, Hamman-Rich syndrome, fulminant necrotizing soft tissue infections (e.g. by S. pyogenes or Panton-Valentine leucocidin-positive S. aureus), fulminant deep tissue infections, fulminant necrotizing fasciitis (Fournier's gangrene), fulminant necrotizing myositis and pyomyositis, fulminant soft tissue pseudotumor, gas gangrene.

Inflammation is a complex biological response of the body towards pathogens, damaged cells, irritants or many other pathologic changes in the organism. There is a pentad of inflammatory signs: Heat, pain, redness, swelling, and loss of function. Fulminant inflammatory diseases comprise, without being limiting, fulminant colitis, fulminant meningitis, fulminant jejunoileitis, Adult Onset Still's Disease (AOSD), granulomatosis with polyangiitis, lymphomatoid granulomatosis, pemphigus, thrombotic thrombocytopenic purpura, fulminant hemophagocytic lymphohistiocystosism, Guillain-Barré syndrome (acute inflammatory demyelinating polyradiculoneuropathy), fulminant myocarditis, fulminant inflammatory leukoencephalopathy, fulminant proliferative vitreoretinopathy, fulminant ocular toxoplasmosis, fulminant sclerosing peritonitis, and others.

Several bacterial infections are a major cause for peracute disease courses. These comprise, without being limiting, sepsis, pneumonic bubonic plague, fulminant bacterial meningitis, meningococcal meningitis, cholera, Weil's disease (leptospirosis infection), fulminant Stenotrophomonas maltophilia soft tissue infection, fulminant bacterial peritonitis, fulminant bacterial endophthalmitis, fulminant Mycoplasma pneumoniae pneumonia, fulminant gram-negative bullous cellulitis, fulminant community-acquired Acinetobacter baumannii infection, fulminant bacterial septicemia, fulminant meningococcemia and hemolytic-uremic syndrome.

Likewise, several viral infections can trigger peracute disease course. These comprise, without being limiting, ebola, Lassa fever, Lábrea fever, rabies, fulminant viral myocarditis or other causes of haemorrhagic or tissue damaging fever.

Several fungal infections can cause peracute disease courses too. These comprise, without being limiting, fulminant fungal rhinosinusitis, Fulminant invasive fungal sinusitis, fulminant non-invasive fungal sinusitis, fulminant fungal sphenoiditis, fulminant mucormycosis, fulminant fungal peritonitis, fulminant fungal meningitis, fulminant fungal cerebritis, fulminant fungal pericarditis, fulminant fungal pneumonia, fulminant fungal meningoencephalitis.

This also holds true for protozoal infections such as primary amebic meningoencephalitis.

Further diseases with a peracute disease course comprise fulminant pre-eclampsia.

There is a variety of fulminant allergic reactions, comprising, without being limiting, asthma attack, acute anaphylaxis, fulminant allergic alveolitis-like hypersensitivity reaction, fulminant allergic purpura.

Another major cause for peracute disease courses are intoxications. Well-known rapidly acting intoxications comprise, without being limiting, intoxication with heavy metals, cyanide, hyperkalemia (e.g. due to potassium chloride injections), colchicine, a variety of venoms from snakes, spiders, centipedes, scorpions, bees, wasps, caterpillars, cone snails, stingrays, sponges, jellyfish, sea anemones, puffer fish, weevers, scorpionfishes, stargazers, salamanders, poison frogs, poison dart frogs, gila monsters, Mexican beaded lizard, shrews, vampire bats.

Also infections with freshwater cyanobacteria and dinoflagellates such as Pfiesteria spec. and Gambierdiscus toxicus can cause a fulminant toxicosis, in particular fulminant liver damages. In particular the lipopolysaccharides from their cell wall, respectively cell membrane can be toxic. Examples for cyanobacterial proteins causing a toxicosis are microcystins such as microcystin-LA (cf. Ibelings and Havens (2008) Adv Exp Med Biol 619: 675-732).

It is understood that upon peracute tissue damage SELENBP1 can only be released into the circulation from tissues in which SELENBP1 is constitutively expressed in a reasonable amount. This holds true for most tissues, but not for all. In detail, expression was found in the choroid, iris, retina, cornea (Okonuki et al. (2007) Exp Eye Res 84:823-831), esophagus (Silvers et al. (2010) Clin Cancer Res 16: 2009-2021), thyroid (Brown et al. (2006) Mol Carcinog 45:613-626; Sofiadis et al. (2012) Eur J Endocrinol 166: 657-667), heart (Chang et al. (1997) J Cell Biochem 64: 217-224), coronary artery (Torrealba et al. (2005) Am J Transplant 5: 58-67), breast lobular units and duct cells (Zhang et al. (2013) PLoS One 8:e63702; Kim et al. (2006) Proteomics 6: 3466-3476), lung (Chang et al. (1997) J Cell Biochem 64: 217-224; Chen et al. (2004) J Pathol 202:321-329; Li et al. (2004) Proteomics 4: 3394-3400), liver (Raucci et al. (2011) Biochim Biophys Acta 1814:513-522; Chang et al. (1997) J Cell Biochem 64: 217-224; Huang et al. (2012) Clin Cancer Res 18:3042-3053; Kim et al. (2006) Proteomics 6: 3466-3476), pancreaticobiliary duct (Kim et al. (2006) Proteomics 6: 3466-3476), kidney (Chang et al. (1997) J Cell Biochem 64: 217-224; Torrealba et al. (2005) Am J Transplant 5: 58-67; Kim et al. (2006) Proteomics 6: 3466-3476), spleen (Chang et al. (1997) J Cell Biochem 64: 217-224), stomach (Xi et al. (2011) Hum Pathol 42: 1620-1628; Zhang et al. (2011) Med Oncol 28: 951-957; Zhang et al. (2011) Med Oncol 28: 481-487; He et al. (2004) Proteomics 4: 3276-3287; Wu et al. (2009) Oncol Rep 21: 1429-1437), colon (Chang et al. (1997) J Cell Biochem 64: 217-224; Pohl et al. (2009) PLoS 4: e7774; Poulsen et al. (2012) BMC Gastroenterol 12:76), adipose tissue (Montes Nieto et al. (2013) J Clin Endocrinol Metab 98: E576-585; McClain et al. (2013) Virchow's Arch 463: 85-92), ovary (Huang et al. (2006) Int J Cancer 118: 2433-2440; Zhang et al. (2010) Hum Pathol 41: 255-261), uterus (Torrealba et al. (2005) Am J Transplant 5: 58-67; Zhang et al. (2010) Diagn Pathol 5: 80), prostate (Yang et al. (1998) Cancer Res 58: 3150-3153; Kim et al. (2006) Proteomics 6: 3466-3476), endothelium (Kim et al. (2006) Proteomics 6: 3466-3476), smooth muscle (Kim et al. (2006) Proteomics 6: 3466-3476; Kim et al. (2006) Proteomics 6: 3466-3476).

It is preferred that the immunoassay according to instant invention refers to a peracute hypoxic event which is selected from the group comprising myocardial infarction, acute coronary syndrome, pulmonary embolism and thrombosis.

It is further preferred that the immunoassay according to instant invention refers to pathologically elevated levels of SelenBP1, which are detectable at the time of the onset of symptoms resulting from the peracute tissue damage.

Up to now, SELENBP1 expression couldn't be shown by microarray analysis or serial analysis of gene expression (SAGE) in white blood cells, tibial nerve, bladder or pituitary.

Therefore, the immunoassay according to the invention is directed only to the detection of peracute tissue damage in at least one of the tissues expressing constitutively SELENBP1, as listed above. Namely, an immunoassay according to the invention is disclosed, wherein the suspected peracute tissue damage has occurred in the choroid, iris, retina, cornea, thyroid, heart, coronary artery, breast lobular units and duct cells, lung, liver, pancreaticobiliary duct, kidney, spleen, stomach, colon, adipose tissue, ovary, uterus, prostate, endothelium, and smooth muscle.

The term “labeling” according to the present invention preferably means labeling by direct or indirect methods. Direct labeling involves coupling of the label directly (covalently or non-covalently) to the molecule to be labeled. Indirect labeling involves binding (covalently or non-covalently) of a second ligand to the molecule to be labeled. Such second ligand should specifically bind to the molecule to be labeled with an at least 3-fold higher, preferably at least 10-fold, and more preferred at least 50-fold higher affinity under assay conditions. Said second ligand may be coupled with a suitable label means and/or may bind a third ligand binding to the second ligand. The use of second, third, or even higher order ligands is often used to increase the signal. Suitable second and higher order ligands may include antibodies, secondary antibodies, and the well-known streptavidin-biotin system (Vector Laboratories, Inc.). Furthermore, the molecule to be labeled or the substrate may also be “tagged” with one or more tags known in the art. Such tags may then be targets for higher order ligands. Suitable tags include biotin, digoxygenin, His-Tag, glutathione-S-transferase, FLAG-tag (N-DYKDDDDK-C), green fluorescence protein (GFP), myc-tag, influenza a virus hemagglutinin (HA), maltose binding protein, and others. In the case of a peptide or polypeptide, the tag is generally located at or close to the N-terminus and/or C-terminus.

Furthermore, the molecule to be labeled or the substrate may also be provided with a suitable “spacer” known in the art in order to avoid in case of bulky molecules any limitations with respect to the binding properties due to spatial constrictions.

The term “labeling means” according to the present invention preferably means any direct or indirect detectable labeling means selected from the group of enzymatic labels, isotopic or radioactive labels, chemiluminescent labels, bioluminescent labels, fluorescent labels, magnetic labels (e.g. “magnetic beads”, including paramagnetic and superparamagnetic labels), dye labels (chromophors), and others known in the art. Suitable labels are detectable by an appropriate detection method known in the art. Suitable labels may further include gold particles, latex beads, acridan ester, luminol, and ruthenium. Preferably suitable are non-radioactive labels.

Enzymatically active labels include, e.g., horseradish peroxidase, alkaline phosphatase, beta-galactosidase, luciferase, and derivatives thereof. Suitable substrates for detection include diamino benzidine (DAB), 3,3′-5,5′-tetramethyl-benzidine, NBT-BCIP (4-nitro blue tetrazolium chloride and 5-bromo-4-chloro-3-indolyl-phosphate, CDP-Star™ (Amersham Biosciences), ECL (Amersham Biosciences) and others known in the art.

A suitable enzyme-substrate combination may result in increase or decrease of a colored reaction product (chromophor), fluorescence, or chemo- or bioluminescence, which can be measured according to methods known in the art (e.g. using a photometer, a photo-multiplier, and a light-sensitive film or camera system). The same principles apply for measuring the endpoint or performance or development of an enzymatic reaction.

Suitable fluorescence labels include fluorescent dyes and proteins (such as GFP and its derivatives), Cy3, Cy5, Texas Red, fluorescein, and the Alexa dyes (e.g. Alexa 568). Further suitable fluorescent labels are commercially available e.g. from Molecular Probes (Oregon, USA). Also the use of quantum dots as fluorescent labels is encompassed. Examples of fluorescent proteins include, but are not limited to, green, yellow, cyan, blue, and red fluorescent proteins.

Suitable chemiluminescence or bioluminescence labels include, but are not limited to prokaryotic (e.g., bacterial lux-encoded) or eukaryotic (e.g., firefly luc-encoded) luciferases, as well as variants possessing varied or altered optical properties, such as luciferases that produce different colors of light, e.g. derived from Photinus pyralis, from the sponge Suberities domuncula, and the Mycena fungi. Furthermore, photoproteins, e.g., calcium-activated photoproteins and their specifically designed variants may be suitable, which are capable of producing light typically in the range of 200 nm to 1100 nm, or in the visible spectrum (i.e., between approximately 350 nm and 800 nm), e.g., obelin from the marine polyp Obelia longissima, or Aequorin, e.g., from the luminescent jellyfish Aequorea victoria or from other organisms may be suitable, optionally in a membrane.

In a preferred embodiment the immunoassay according to the invention is a spectro-photometric immunoassay.

Suitable radioactive labels include <35>S, <125>I, <32>P, <33>P and the like. A radioactive label can be detected by any method known and appropriate, e.g. a light-sensitive film or a phosphor imager.

Suitable detection methods according to the present invention also include precipitation (particularly immunoprecipitation), electrochemiluminescence (electrically generated chemiluminescence), bioluminescence, RIA (radioimmunoassay), ELISA (enzyme-linked immunosorbent assay), sandwich enzyme immune tests, sandwich immunoassays (ECLIA), dissociation-enhanced lanthanide fluorescent Immunoassay (DELFIA™, PerkinElmer Inc., USA), CBA (cobalt-binding assay), scintillation proximity assay (SPA), turbidimetry, nephelometry, latex-enhanced turbidimetry or nephelometry, latex agglutination assay, solid phase immune assays and the like.

Further methods known in the art (such as gel electrophoresis, 2D gel electrophoresis, SDS polyacrylamid gel electrophoresis (SDS-PAGE), Western Blotting, and mass spectrometry), can optionally be used in combination with the labeling or other detection methods as described above.

The one or more antigenic molecules as defined in the present invention may be directly or indirectly provided with the labeling means, substantially as hereinbefore described.

The term “second labeling means” according to the present invention preferably means any direct or indirect detectable labeling means selected from the group of chemiluminescent labels, bioluminescent labels, fluorescent labels, dye labels (chromophors), and others known in the art. The use of the term “second labeling means” preferably means that the second labeling means is not identical with the aforementioned (primary or first) labeling means. Suitable second labeling means are detectable by an appropriate detection method known in the art based on resonance energy transfer (RET) principle. Preferably, second labeling means include labeling means suitable for fluorescence resonance energy transfer (FRET), bioluminescence resonance energy transfer (BRET), or chemiluminescence resonance energy transfer (CRET) as known to the person skilled in the art. The selection of a suitable second labeling means will consider the kind of the first labeling means in order to safeguard that the RET signal is detectable and are known to the person skilled in the art. Furthermore, the RET signal generation and selection of suitable labeling means provides the person skilled in the art with additional information about the kind and kinetics of complex formation and the structural features of the complexes formed.

In a preferred embodiment the immunoassay according to the invention is carried out as a rapid or point-of-care test.

A further aspect of the invention is the use of an antibody binding to at least one epitope of SELENBP1 in a test sample from a mammal including humans, wherein said test sample is suspected to display a pathologically elevated level of SELENBP1, for determining the degree of peracute tissue damage in said mammal.

The term “kit” according to the present invention preferably means a collection of the aforementioned means to perform the method of the invention, preferably, provided in separately or within a single container. The container preferably comprises user instructions for carrying out the method according to the present invention.

In yet another aspect a kit is disclosed that comprises an antibody binding to at least one epitope of SELENBP1.

It is preferred that in the kit according to the invention said antibody has a detection sensitivity, respectively detection limit of SELENBP1 of 0.10 nmol/l in a test sample or lower.

The kit according to the invention comprises additionally at least one standard comprising SELENBP1 for calibration purposes.

In another embodiment said kit according to the invention comprises additionally at least one antibody against Troponin T and/or Troponin I and optionally comprises additionally at least one Troponin T and/or Troponin I calibration standard.

It is preferred that the kit according to the invention is suitable for performing the immunoassay according to the invention in an automated analyzer, by means of a test strip or disc, or as a rapid test.

### EXAMPLES

All statistical analyses were performed using freely available statistical software (R 3.5.1, The R Foundation for Statistical Computing). When testing for differences between groups, Wilcoxon's rank-sum test was used. Confidence intervals (CI) and significance were reported for an interval of 0.95 and accordingly an a of 0.05. Room temperature (RT) in the following examples means 22° C. If not otherwise stated “%” means % w/w.

### Example 1: SELENBP1 can be Detected in Blood Samples from Patients with Acute Coronary Syndrome

Circulating SELENBP1 was measured in serum samples from patients suspected to suffer from Acute Coronary Syndrome (ACS). The diagnosis was confirmed in parallel by traditional methods. The blood samples had been drawn at several time points after first onset of symptoms suspected to be due to ACS. Average SELENBP1 concentrations of patients were higher than in healthy controls. The concentrations showed a dynamic time course with patient-specific characteristics.

### Example 2: SELENBP1 in Blood of Healthy Controls

High quality serum samples from a small group of control adult (n=75) were used to test normal SELENBP1 levels in the blood of healthy subjects. Only marginal concentrations were detectable, mainly below the linear range of the SELENBP1 assay. The values obtained were normally distributed with a mean of 0.23 nmol/l. The 95% percentile of the values of SELENBP1 concentrations of the healthy control group were up to 0.32 nmol/l, the 99% percentile up to 0.39 nmol/l.

### Example 3: Expression and Purification of Recombinant SELENBP1

Recombinant human SELENBP1 (rhSELENBP1) was expressed in baculovirus-infected insect cells. The cDNA sequence encoding rhSELENBP1 (according to UniProtKB database No. Q13228-1) was amplified by PCR from hepatic cDNA according to SEQ:ID NO. 1 using primers P1 (SEQ:ID NO. 2) and P2 (SEQ:ID NO. 3) containing a BamHI and a HindIII restriction site, respectively (Invitrogen, Thermo Fischer Scientific, Dreieich, Germany). The pFastBac1 plasmid (Thermo Fischer Scientific) was digested with BamHI and HindIII, the plasmid fragment was removed and replaced with the PCR sequence giving rise to pFastBac2-SelenBP1-His6 plasmid.DH10Bac E. coli cells were transformed and Bacmid-positive cells were identified, cultivated and recombinant bacmid was isolated. Sf9 insect cells were transferred with bacmid DNA by Cellfectin (Thermo Fischer Scientific) for obtaining a recombinant virus stock that was used to initiate rhSELENBP1 biosynthesis in “High Five” insect suspension cells. 72 h after, infection cells were harvested, lysed and rhSELENBP1-His6 was isolated from cell extract using affinity chromatography on Ni-NTA agarose according to the manufacturer's instructions (Qiagen GmbH, Hilden, Germany). The concentration of purified rhSELENBP1 was determined using a commercial bicinchoninic acid (BCA) protein assay kit (Pierce BCA, Thermo Fischer Scientific).

### Example 4: Immunization, Isolation and Purification of Antibodies Against SELENBP1

Monoclonal antibodies (mAb) were generated essentially as described (Hybsier et al. (2017) Redox Biology 11: 403-414) by a commercial service provider (UNICUS Karlsburg OHG, Greifswald, Germany). In brief, BALB/c mice were immunized with an emulsion of purified rhSelenBP1 in TiterMax® Gold adjuvant (Sigma-Aldrich Corp., St. Louis, USA), followed by additional injections for boosting the immune response. Antibody titers were determined by an indirect ELISA with immobilized rhSELENBP1 in combination with polyclonal rabbit anti-mouse antibodies. Positive mice were sacrificed, the splenocytes isolated, fused with myeloma cells and hybridomas were selected in hypoxanthine-aminopterin-thymidine (HAT) medium. Positive cultures were propagated and re-cloned by limiting dilution to obtain homogenous cell clones. Secreted antibodies were purified by standard methods using protein A chromatography by a commercial service supplier (InVivo Biotech Services, Hennigsdorf, Germany).

### Example 5: Assay Development and Characterization

For quantitative analysis of SELENBP1 several mAb combinations were compared and one suitable pair of clones was selected for mAb production and purification, essentially as described (Hybsier et al. (2017) Redox Biology 11: 403-414). Two suitable mAb (anti-SELENBP1-mAb1 and anti-SELENBP1-mAb2) were chosen and a two-site-non-competitive immunoassay (sandwich assay) was established. Phosphate buffered saline (PBS) with 0.05 M KH₂PO₄, 0.1 M NaCl, adjusted to pH 6.5 was used as a basis. For coating purposes flat-bottomed, white high binding 96-well plates (Greiner Bio-One, Frickenhausen, Germany) were incubated with 100 μl PBS containing 2.1 μg/ml anti-SELENBP1-mAb1 per well for 12 h at 4° C. Calibration standards were generated by diluting the rhSELENBP1 preparation in PBS containing 1.0 mmol/l BSA. A set of positive standards was prepared by adding rhSELENBP1 to a human serum sample.

The assay parameters were optimized for improving the signal/noise ratio, and the following protocol was routinely used, if not indicated otherwise. The plates were washed four times using PBS with 1% (v/v) Triton X-100. Next, 85 μl PBS with 10% (v/v) glycerol and 1% (w/v) BSA and 15 μl sample per well were added, the plates were sealed and placed on a microplate shaker for 1 h at room temperature. Thereafter, the plated were washed four times with PBS/BSA and incubated with 100 μl PBS with 0.05% (v/v) MACN-labelled anti-SELENBP1-mAb2, 0.1% (v/v) Triton X-100 and 5% (w/v) skim milk powder per well and, again, placed on a shaker for another 1 h at room temperature.

Finally, the plates were washed again four times with PBS/BSA and placed into a luminometer (Mithras LB 940, Berthold, Bad Wildbad, Germany) for analysis. Detection of SELENBP1 was accomplished photometrically, after injecting 75 μl of 0.06% (v/v) H₂O₂ and 0.2 M NaOH to induce light emission at 430 nm. Relative light units (RLU) were recorded for 1 s. Six calibration standards with increasing rhSELENBP1 concentrations were included in each plate, and SELENBP1 concentrations were calculated by linear regression as stated below. In addition, each plate contained a standard serum prepared with 4.8 nmol/l rhSELENBP1. This standard allows a comparison of the accuracy of this novel luminometric immunoassay (LIA) across different plates (inter assay variation).

The standards used for analyzing clinical serum samples ranged from 0.3 to 38.2 nmol/l rhSELENBP1. Quantification was based on linear regression of the RLU as a function of SELENBP1 concentration, with m and b being plate-specific parameters:

SELENBP1=e^{(ln(RLU)-b)/m}

Concentrations below the linear measuring range were linearly extrapolated.

### Example 6: Sensitivity

Different quantities of rhSELENBP1 were analyzed in order to assess functional assay sensitivity (FAS) of the LIA. Each concentration was measured in triplicate. All samples were prepared with 1.2 mmol/l BSA to mimic regular concentration of serum protein. FAS was defined as the range of rhSELENBP1 concentrations allowing reliable measurements with coefficients of variation (CV) below 20%.

### Example 7: Stability of SELENBP1 in Serum

Handling and storage of serum samples are major issues in daily hospital routine as well as during scheduled analysis of blood samples received from patients enrolled in clinical trials. It is thus important to determine the stability of the analyte in a given matrix at different temperatures and upon freezing and thawing, respectively.

Stability of SELENBP1 was analyzed by exposing serum samples to prolonged storage at room temperature or at 4° C., and by repeated cycles of freezing and thawing, respectively. Two individual serum samples were drawn using BD Vacutainer systems with SST tubes (Becton Dickinson GmbH, Heidelberg, Germany), left at room temperature for 30 min to allow coagulation, and centrifuged at 2600 rpm for 10 min at room temperature to separate serum from the clotted material. Three preparations were made from the supernatant of the first serum sample: One left as is (E), a second and a third sample supplemented with rhSELENBP1 to 5.7 nmol/l (C) and 15.3 nmol/l (D), respectively. The supernatant of the second serum sample was left as is (A) or supplemented with rhSELENBP1 to 5.7 nmol/l (B). Six aliquots were prepared from each of the preparations A-E and placed at 4° C. or room temperature. Aliquots were taken after 1 h, 3 h, 6 h, 1 d, 3 d and 7 d, respectively. The aliquots were stored at −20° C. until analysis. Ten more aliquots were taken of the preparations B, D and E for the determination of stability upon repeated freeze-thawing cycles.

Storage at 4° C. for up to 7 h had no effect on the concentration of SELENBP1 in serum, whereas a 7 h storage period at room temperature reduced immunoreactive SELENBP1 to 73% (58-88%) of control.

In contrast to prolonged incubation times at room temperature, freezing had little influence, and a marginal decline to 81% (72-90%) of initial SELENBP1 concentrations was observed after 10 cycles of freezing and thawing.

### Example 8: Intra- and Inter-Assay Variability

A duplicate of samples from a standard serum with added rhSELENBP1 was analyzed with each 96 well plate in duplicates and used to calculate the CV between plates (inter-assay CV). To assess variability within duplicates the quantities of the CVs were calculated.

The inter-assay CV was determined with a set of standards of rhSELENBP1 in a total of 32 analyses. The average concentration of the standards was 4.6 nmol/l (4.4-4.7 nmol/l). The average inter-assay CV was 9.9%.

To assess intra-assay variability, 1394 duplicates were analyzed and the 5^{th}, 50^{th} and 95^{th} quantile of the CVs were calculated. This yielded a CV of 0.2%, 3% and 12%, respectively.

### Example 9: Comparison of Analytes

Different matrices were tested for their impact on the measurement and its precision.

rhSELENBP1 was added to freshly drawn serum, as well as to citrate-, heparin- and EDTA-plasma samples. After incubation at room temperature, SELENBP1 concentrations were determined by the luminometric immunoassay as described above.

Variability of the results between the matrices was low (mean CV: <12.5%). This indicates that all matrices tested are suitable for SELENBP1 quantification.

### Example 10: SELENBP1 does not Coincide with Other Blood Biomarkers for Tissue Damage

In order to compare the kinetics of SELENBP1 release into the blood by peracute tissue damage with established biomarkers the dynamics and specificity of its concentrations have been compared to Troponin T (high-sensitivity assay, hsTnT) as a marker for myocardial tissue damage, and to ASAT (aspartate aminotransferase) indicating muscle, liver, or heart damage. None of these biomarkers showed a significant correlation to SELENBP1 concentrations. This indicates that the release of SELENBP1 from damaged tissue into the blood is unrelated to the extent of myocardial necrosis.

Similarly, no correlation was found between circulating SELENBP1 and creatinine, potassium, glucose, hemoglobin, fibrinogen, prothrombin time, platelet counts, cholesterol or white blood cell counts.

Sequences:

SEQ: ID No. 1: atg gctacgaaatgtgggaattg tggacccggc tactccaccc
ctctggaggc catgaaagga cccagggaag agatcgtcta cctgccctgc atttaccgaa
acacaggcac tgaggcccca gattatctgg ccactgtgga tgttgacccc aagtctcccc
agtattgcca ggtcatccac cggctgccca tgcccaacct gaaggacgag ctgcatcact
caggatggaa cacctgcagc agctgcttcg gtgatagcac caagtcgcgc accaagctgg
tgctgcccag tctcatctcc tctcgcatct atgtggtgga cgtgggctct gagccccggg
ccccaaagct gcacaaggtc attgagccca aggacatcca tgccaagtgc gaactggcct
ttctccacac cagccactgc ctggccagcg gggaagtgat gatcagctcc ctgggagacg
tcaagggcaa tggcaaaggg ggttttgtgc tgctggatgg ggagacgttc gaggtgaagg
ggacatggga gagacctggg ggtgctgcac cgttgggcta tgacttctgg taccagcctc
gacacaatgt catgatcagc actgagtggg cagctcccaa tgtcttacga gatggcttca
accccgctga tgtggaggct ggactgtacg ggagccactt atatgtatgg gactggcagc
gccatgagat tgtgcagacc ctgtctctaa aagatgggct tattcccttg gagatccgct
tcctgcacaa cccagacgct gcccaaggct ttgtgggctg cgcactcagc tccaccatcc
agcgcttcta caagaacgag ggaggtacat ggtcagtgga gaaggtgatc caggtgcccc
ccaagaaagt gaagggctgg ctgctgcccg aaatgccagg cctgatcacc gacatcctgc
tctccctgga cgaccgcttc ctctacttca gcaactggct gcatggggac ctgaggcagt
atgacatctc tgacccacag agaccccgcc tcacaggaca gctcttcctc ggaggcagca
ttgttaaggg aggccctgtg caagtgctgg aggacgagga actaaagtcc cagccagagc
ccctagtggt caagggaaaa cgggtggctg gaggccctca gatgatccag ctcagcctgg
atgggaagcg cctctacatc accacgtcgc tgtacagtgc ctgggacaag cagttttacc
ctgatctcat cagggaaggc tctgtgatgc tgcaggttga tgtagacaca gtaaaaggag
ggctgaagtt gaaccccaac ttcctggtgg acttcgggaa ggagcccctt ggcccagccc
ttgcccatga gctccgctac cctgggggcg attgtagctc tgacatctgg atttga
Seq ID No. 1 corresponds to the open reading frame encoding
SELENBP1 (UniProt KB Entry No. Q13228-1)
SEQ: ID NO. 2: atcggatccaccatggctacgaaatgtgggaattgtg (Primer P1)
SEQ: ID NO. 3: atcaagcttcagtgatggtgatggtgatgaatccagatgtcagagctaca
atcgcc (Primer P2)

1. An immunoassay for determining peracute tissue damage in a test sample from a subject suspected thereof, the immunoassay comprising the steps of:
a) contacting a test sample taken from a subject suspected to suffering from peracute tissue damage with an antibody that binds specifically to at least one epitope of selenium binding protein 1 (SELENBP1) to form an antibody:SELENBP1 complex;
b) detecting the amount of antibody:SELENBP1 complex in said test sample; and
c) assessing the degree of peracute tissue damage based on the detected amount of antibody:SELENBP1 complex.

2. The immunoassay according to claim 1 or 2, wherein said test sample is selected from a body fluid selected from the group consisting of blood, plasma, serum, urine, and liquor.

3. The immunoassay according to claim according to claim 2, wherein peracute tissue damage is determined if the amount of SELENBP1 calculated from the detected amount of antibody:SELENBP1 complex in said sample obtained from said individual, which is selected from blood, plasma and serum is higher than 0.32 nmol/l.

4. The immunoassay according to claim according to claim 2, wherein peracute tissue damage is determined if the amount of SELENBP1 calculated from the detected amount of antibody:SELENBP1 complex in said sample obtained from said individual, which is selected from urine and liquor is higher than 0.1 nmol/l.

5. The immunoassay according to claim 1, wherein said immunoassay uses a monoclonal antibody that binds to at least one epitope of SELENBP1.

6. Use of an antibody binding to at least one epitope of SELENBP1 in a test sample from a mammal including humans, wherein said test sample is suspected to display a pathologically elevated level of SELENBP1, for determining the degree of peracute tissue damage in said mammal.

7. A kit for the performance of the immunoassay as defined in claim 1 comprising an antibody binding to at least one epitope of SELENBP1.

8. The kit according to claim 7, wherein the detection limit of said immunoassay for SELENBP1 in a test sample calculated from the detected amount of antibody:SELENBP1 complex is 0.10 nmol/l.

9. The kit according to claim 7, comprising additionally at least one SELENBP1 calibration standard, and optionally comprising additionally at least one antibody against Troponin T and/or Troponin I, and optionally comprising additionally at least one Troponin T and/or Troponin I calibration standard.

10. The kit according to claim 7 for performing the immunoassay as defined in claim 1 in an automated analyzer, by means of a test strip, test disc or as a rapid or point-of-care test.
